(19) United States
(12) Patent Application Publication
(10) Pub. No.: US 2014/0180120 A1
(43) Pub. Date: Jun. 26, 2014

(54) Ultrasound Imaging Catheters with Reciprocating Phased Array Transducers

(71) Applicant: Volcano Corporation, San Diego, CA (US)

(72) Inventor: Norman H. Hossack, Folsom, CA (US)

(73) Assignee: Volcano Corporation, San Diego, CA (US)

(21) Appl. No.: 14/135,270

(22) Filed: Dec. 19, 2013

Related U.S. Application Data
(60) Provisional application No. 61/745,360, filed on Dec. 21, 2012.

(51) Int. Cl. A61B 8/00; A61B 8/12

(52) U.S. Cl. CPC A61B 8/4488; A61B 8/12
USPC 600/463; 600/467

(57) ABSTRACT

In one embodiment, a catheter imaging device includes an elongated catheter shaft having a forward looking ultrasonic transducer disposed at a distal end thereof. A first device is coupled to the transducer for sweeping an ultrasonic beam produced by the transducer in a first plane and through a first angle therein, and a second device is coupled to the transducer for sweeping the ultrasonic beam in a second plane and through a second angle therein, the second plane being disposed orthogonal to the first plane. In one aspect, the ultrasonic transducer includes a plurality of transducer elements.

### CROSS-REFERENCE TO RELATED APPLICATION

The present application claims the benefit of the filing date of provisional U.S. Patent Application No. 61/745,360 filed Dec. 21, 2012. The entire disclosure of this provisional application is incorporated herein by this reference.

### BACKGROUND

1. Technical Field

The present disclosure relates to medical imaging devices in general, and in particular, to ultrasound catheters having reciprocating, forward looking, phased array transducers.

2. Related Art

The development of new medical technologies and equipment has provided an increasing number of options to physicians for the diagnosis and treatment of cardiovascular diseases. For example, ultrasound imaging technologies have enabled doctors to create and view a variety of images generated by sensors inserted within a blood vessel.

One ultrasound imaging technology that has been employed to good effect is intravascular ultrasound (IVUS). In IVUS imaging systems, an ultrasonic transducer assembly is attached to a distal end of a catheter. The distal end of the catheter is carefully maneuvered through a vessel in a patient's body, usually by means of a guide wire, to an area of interest, such as within a coronary artery. The transducer assembly transmits focused ultrasound pulses and receives echoes of those pulses that are reflected back from adjacent tissue and other structures and delayed in time. The echoes received by the transducer are then converted to electrical signals and transmitted to processing equipment where, for example, they can be converted to an image of the area of interest that can be displayed.

Intracardiac echocardiography (ICE) is yet another imaging technology that has also been used to good effect. ICE is similar to IVUS in that it uses a catheter with a transducer assembly at its distal end to facilitate imaging. However, ICE involves maneuvering or “steering” the tip of the catheter, and hence, the transducer assembly, into the heart, typically under fluoroscopy, so that the chamber walls and other structures of a heart can be imaged. ICE catheters also typically include a steering mechanism that enables articulation of the distal end of the catheter for such purposes.

IVUS and ICE catheters are necessarily relatively small in size because they need to be capable of traversing the lumen of a vein or artery. Consequently, the transducer assembly needs to be correspondingly small, while at the same time, constructed to provide an imaging area that is as large as possible.

A forward looking ultrasound catheter device with a transducer that pivots reciprocally through an angle (i.e., of either elevation or azimuth) relative to the longitudinal axis of the catheter to form a two-dimensional (2D), fan-shaped “slice” through the area of interest is described in commonly owned U.S. Pat. No. 8,317,713 to S. Davies, et al., the disclosure of which is incorporated herein by reference. However, the transducer used in this catheter is “monolithic,” i.e., incorporates only a single transducer element that emits a beam in only one direction. Accordingly, the catheter must be manually rotated about its longitudinal axis in order to gather three-dimensional (3D) image data.

Another known way of implementing an ultrasound imaging catheter is to utilize a “side-looking” transducer element or linear array at the distal end of the catheter, wherein the transducer or array is pivoted reciprocally about the longitudinal axis of the catheter, as described in U.S. Pat. No. 8,317,711 to P. Dala-Krishna, the disclosure of which is incorporated herein by reference. Another side-looking catheter device includes a transducer in which a linear array of transducer elements is disposed circumferentially around the longitudinal axis of the catheter, as described in commonly owned U.S. Pat. No. 7,846,101 to M. Eberle, et al., the disclosure of which is incorporated by reference. However, such transducer arrangements are capable of providing only side-looking views, and are incapable of imaging a relatively large area directly ahead of or distal to the catheter.

Accordingly, a long-felt but as yet unsatisfied need exists in the medical imaging field for a forward looking, ultrasonic IVUS or ICE catheter that is capable of capturing 3D image data sets without having to be manually rotated about its longitudinal axis.

### SUMMARY

In accordance with embodiments of the present disclosure, forward looking, ultrasonic IVUS or ICE catheter devices that are capable of capturing 3D conical image data sets without being manually rotated about their longitudinal axes are provided, together with methods for making and using them therapeutically and diagnostically.

In one example embodiment, a catheter imaging device includes an elongated catheter shaft having a forward looking ultrasonic transducer disposed at a distal end thereof. A first device is coupled to the transducer for sweeping an ultrasonic beam produced by the transducer in a first plane and through a first angle therein, and a second device is coupled to the transducer for sweeping the ultrasonic beam in a second plane and through a second angle therein, the second plane being orthogonal to the first plane. In some embodiments, the transducer can comprise a linear phased array transducer and one of the two sweeping devices can comprise circuitry for sweeping the ultrasonic beam electronically.

In another example embodiment, a method for acquiring a page of three-dimensional (3D) image data of a selected field of view of, for example, an interior of a body vessel, cavity or chamber, using the catheter device above comprises sweeping the ultrasonic beam through the first angle in the first plane using the first device to form a first frame of two-dimensional (2D) image data, incrementing the angular position of the beam by a selected amount in the second plane using the second device, sweeping the beam through the first angle in the first plane using the first device to form a second frame of two-dimensional (2D) image data spatially adjacent to the first frame, repeating the preceding steps until the entire field of view has been scanned by the beam, and combining the 2D image data frames to form a page of 3D image data of the subject field of view.

The scope of the present disclosure is defined by the claims appended hereafter, which are incorporated into this section by reference. A more complete understanding of the features and advantages of the novel ultrasonic catheter devices of the disclosure and the methods for making and using them will be afforded to those skilled in the art by a consideration of the detailed description of some example embodiments thereof presented below, particularly if such consideration is made in conjunction with the appended drawings, wherein like reference numerals are used to identify like elements illustrated in one or more of the figures thereof.

### BRIEF DESCRIPTION OF THE SEVERAL VIEWS OF THE DRAWINGS

FIG. 1 is an illustration of an example embodiment of an ultrasonic catheter imaging system in accordance with the present invention, including a forward looking ultrasound catheter device and an associated control, processing and display module thereof.

FIG. 2 is a functional block diagram of an example embodiment of a control and processing module of the example ultrasonic catheter imaging system of FIG. 1.

FIG. 3 is an upper, left-side perspective view of an example embodiment of a transducer assembly of the example catheter device of FIG. 1.

FIG. 4A is a partial cross-sectional side elevation view of the example transducer assembly of FIG. 3.

FIG. 4B is a partial cross-sectional side elevation view of an alternative embodiment of a transducer assembly.

FIG. 5 is a front end elevation view of the example transducer assembly of FIG. 3, wherein elements of the transducer have been omitted to reveal details of a reciprocating transducer pivoting mechanism.

FIG. 6 is a cross-sectional view of the example transducer assembly of FIG. 3, as seen along the lines of the section A-A taken in FIG. 4A.

FIG. 7 is a plan view illustrating a two-dimensional sweep pattern of the transducer of the example transducer assembly of FIG. 3, as seen in either an azimuthal or an elevation plane.

FIG. 8 is a perspective view illustrating a three-dimensional sweep pattern of the transducer of the example transducer assembly, showing the sweep of the transducer in both the azimuthal and the elevation planes.

FIGS. 9A-9F are images taken sequentially in the lumen of a vessel of a body during an example “fly-through” procedure performed using an ultrasound catheter having a reciprocating, forward looking, phased array transducer in accordance with the present invention.

### DETAILED DESCRIPTION

In embodiments of the present disclosure, ultrasonic catheter imaging systems are provided, together with methods for making and using them. Embodiments of the system include a forward looking, ultrasonic IVUS or ICE catheter device that is capable of capturing conical 3D image data sets without having to be rotated about its longitudinal axis. For the purpose of illustration, the ultrasound catheter device described in the context of an ultrasound catheter system for use as an intracardiac echocardiography (ICE) catheter or an intravascular ultrasound (IVUS) catheter. However, it should be understood that other applications of the disclosed catheter device are contemplated for alternative embodiments. Indeed, the disclosed ultrasound catheter device can be used in any application where it is desirable to image an anatomical chamber or cavity that is accessible via a lumen. For example, although disclosed in the context of ultrasound imaging, it should be appreciated that other imaging techniques, including light-based imaging, can utilize the structures and methods of the present disclosure.

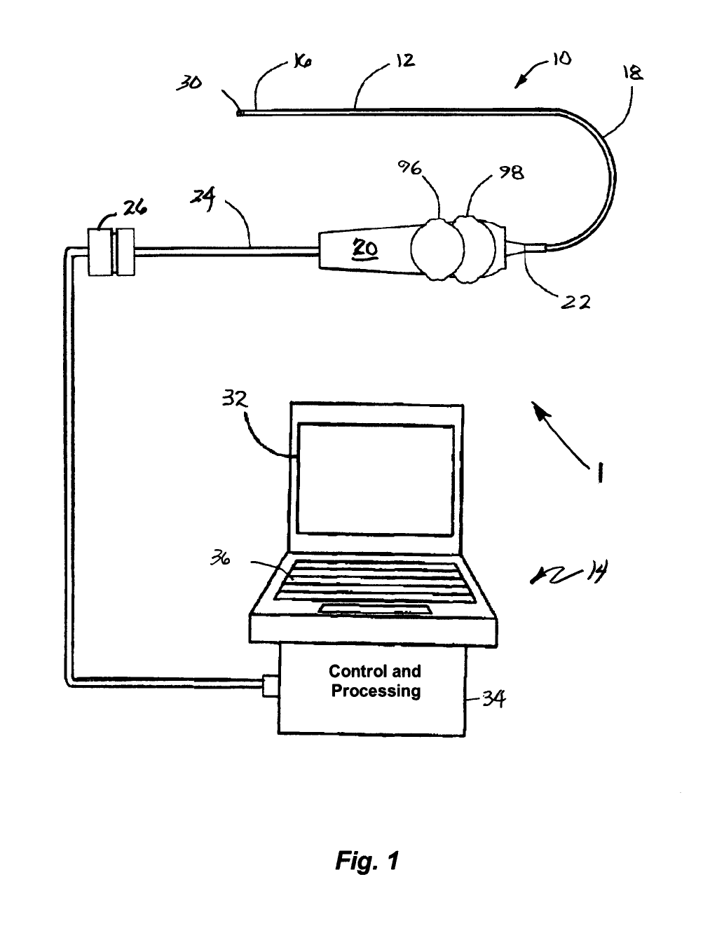

FIG. 1 is an illustration of one example embodiment of an ultrasonic catheter imaging system 1 in accordance with the present invention, including a forward looking ultrasound catheter device 10 that includes an elongated catheter shaft 12, and an associated control, processing and display module 14 thereof. The catheter shaft 12 is a generally flexible, elongated annular member having a distal segment 16 and a proximal segment 18. Optionally, the proximal segment 16 can be attached to a handle 20, e.g., by a resilient strain reliever 22, for manipulation of the catheter shaft 12 and manual control of the catheter device 10. In some embodiments, the handle 20 can include, for example, a motor, e.g., a stepping motor, arranged to rotate a drive shaft disposed in the catheter shaft 12, and/or can incorporate one or more actuators for steering the catheter shaft 12 in accordance with the description that follows.

In some embodiments, the handle 20 can include an electrical interconnection cable 24 with an electrical connector 26 disposed at a proximal end of thereof. As illustrated in the example embodiment of FIG. 1, the cable 24 and connector 26 can be used to interconnect the catheter device 10 with the associated example control, processing and display module 14 shown therein for processing, storing, manipulating and/or displaying image data obtained from signals generated by a forward looking ultrasound transducer assembly 30 mounted at the distal end of the distal segment 16 of the catheter device 10.

As illustrated in FIG. 1, in some embodiments, the associated control, processing and display module 14 of the imaging system 1 can include a display module 32, e.g., an LCD panel or the like, a system control and processing module 34, and one or more input devices 36, such as the keyboard illustrated. Other known types of command and control devices 36 (not illustrated) that can also be used advantageously in the imaging system 1 include, for example, a computer mouse, joystick, pointing pen, touch screen, and the like.

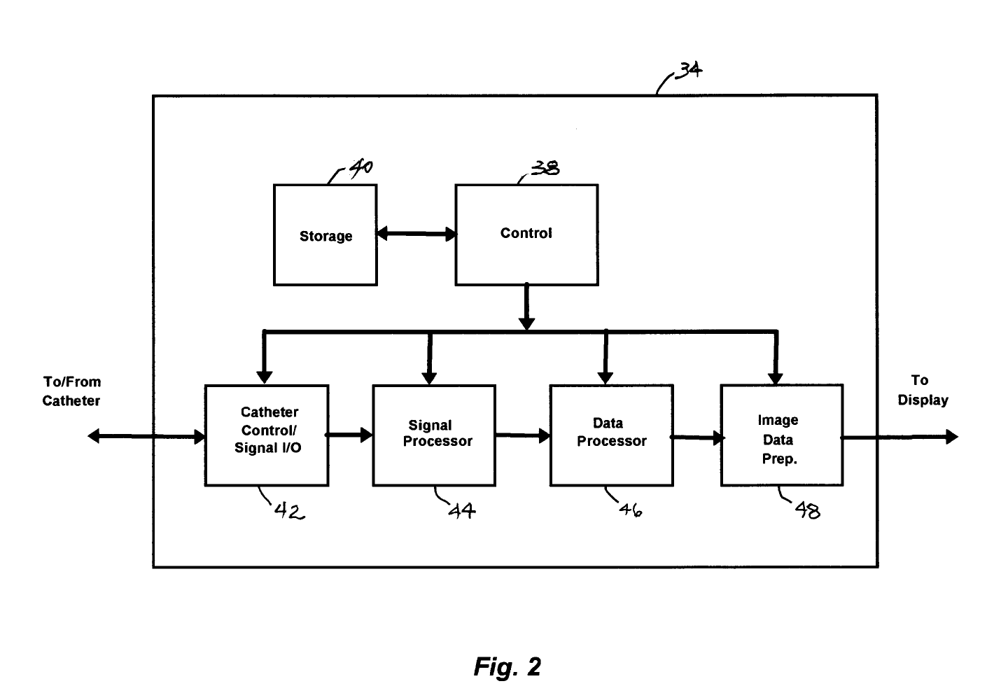

As illustrated in the functional block diagram of FIG. 2, in only one of many possible implementations, the system control and processing module 34 can include a programmable central command and control module 38, a data storage module 40, a catheter device 10 control and signal input and output module 42, a signal processing module 44, a data processing module 46, and an image data preparation, or “rendering” module 48.

As those of some skill will understand, control of the catheter device 10 can include control of both the direction and power of the ultrasound “beam” output by the transducer assembly 30 of the catheter device 10, as well as the timing thereof, since the same transducer assembly 30 is used to “listen” for reflections of the beam from distant objects and convert those to electrical signals that can in turn be converted to image data.

As illustrated in FIG. 2, in some embodiments, the direction, power and timing of the beam can be effected by the catheter control and signal I/O module 42 under the control of the central command and control module 38. Additionally, in some embodiments, the electrical signals output by the transducer assembly 30 can be converted to 3D data images by the signal processor 44, and manipulation of this data to form various image “slices,” i.e., various 2D data images, can be performed by the data processor module 46. These data images can be rendered for display on the display module 32 by the image rendering module 48. However, it should be understood that, in other embodiments, at least some of the above functions of the system control and processing module 34 can be distributed to other components of the imaging system 1, for example, to the handle 20.

In only one of many possible embodiments, many of the functions of the system control and processing module 34, including those of the data processing and rendering modules 46 and 48, can be implemented in a manner similar to that described in U.S. Pat. No. 7,612,773 to P. Magnin, et al., the disclosure of which is incorporated herein by reference.

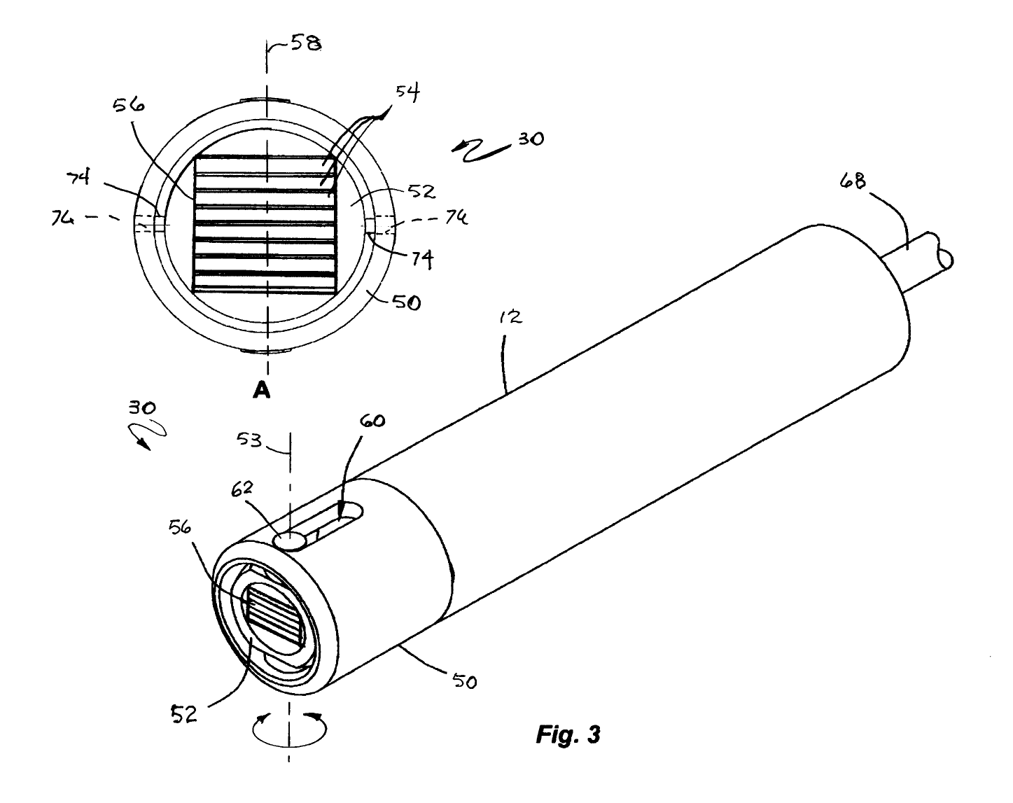

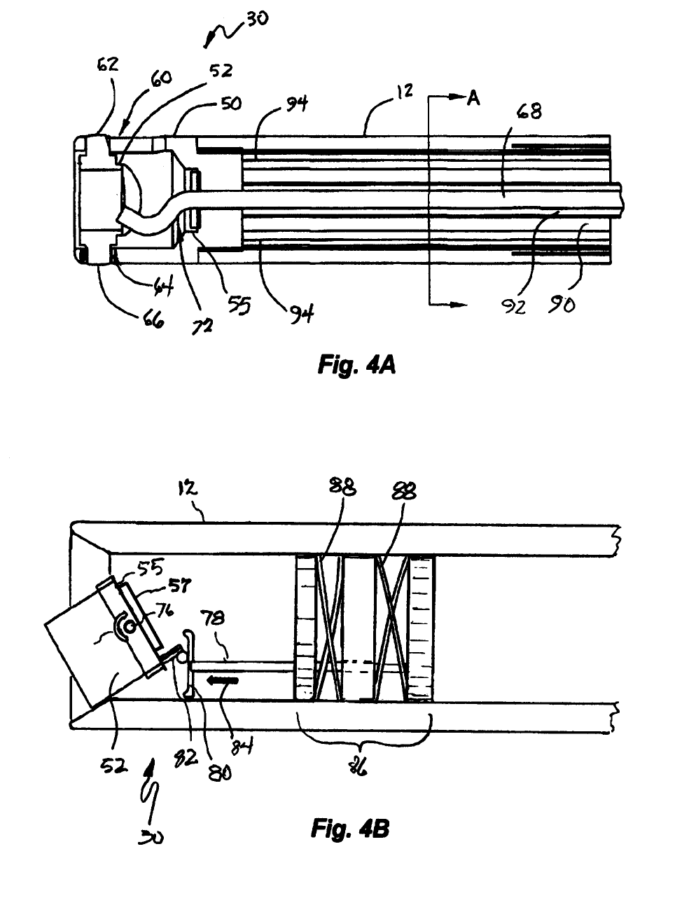

FIG. 3 is an upper, left-side perspective view of an example embodiment of a transducer assembly 30 located at the distal end of the example catheter device 20 of FIG. 1, and FIG. 4A is a partial cross-sectional side elevation view thereof. As can be seen in FIGS. 3 and 4A, the example transducer assembly 30 includes an annular collar 50 coupled to the distal end of the shaft of the catheter 12, and a transducer 52 is disposed concentrically within the collar 50 for left/right reciprocating pivotal movement in a transverse, or horizontal plane of the transducer assembly 30, i.e., about a vertical axis 53 that is substantially transverse to the longitudinal axis of the catheter shaft 12.

As illustrated in the enlarged front end elevation detail view “A” of FIG. 3, the transducer 52 can comprise a plurality of long, narrow, rectangular transducer elements 54 disposed in a stacked arrangement to define a one-dimensional, or “linear” phased array 56. As is known, the elements 54 can be separately pulsed with voltages whose phases are controlled in such a way as to cause the array 56 to emit a focused “beam” of ultrasonic sound energy in a vertical or sagittal plane 58 of the transducer 52 that can be “steered,” or directed, up and down in selected directions within the sagittal plane 58. In some embodiments, a bundle of individual conductive wires (not illustrated) can convey the respective phased actuation voltages through the handle 20 and catheter shaft 12 and to respective ones of the transducer elements 54, and can convey the electrical signals produced by the array 56 in response to its receipt of reflected ultrasound signals back through the catheter 12 and handle 20, and thence, to the control, processing and display module 14.

Each of the transducer elements 54 can comprise a cuboid (or other suitable shape) of a piezoelectric material, e.g., lead zirconate titanate (PZT), that is sandwiched between a pair of conducting electrodes. Each transducer element 54 also preferably includes a backing material (not illustrated) of such type and characteristic as to minimize the effects of “ringing” in the transducer 52 caused by the absorption and/or scattering of spurious echoes. Some examples of the types of transducer elements 54 that can be used in the transducer 52 illustrated are shown and described in commonly owned U.S. Pat. No. 5,368,037 M. Eberle, et al., which is incorporated herein by reference.

In the particular example embodiment shown in FIGS. 3 and 4A, the collar 50 includes an opening, e.g., a longitudinal slot 60, in the upper surface of its distal end that is configured to receive a first pin 62 extending vertically upward from the transducer 52, and forming a part of a “Scotch yoke” mechanism described below that is utilized both to retain the transducer 52 within the collar 50 and to effect reciprocating pivotal movement of the transducer 52 in the transverse plane, as described in more detail below. As illustrated in FIG. 4A, the collar 50 can further includes an aperture 64 in its lower distal end that is configured to receive a second pin 66 extending vertically downward from the transducer 52. The pins 62 and 66 and their respective openings and apertures 60 and 60 act as journals and bearings to enable pivotal movement of the transducer 52 in the transverse plane of the transducer assembly 30 and about the vertical axis 53 defined by the two pins 62 and 66.

In some embodiments, the slot 60 and aperture 66 can cooperate with the first second pins 62 and 66 to facilitate manufacture of the transducer assembly 30, and more particularly, insertion of the transducer 52 into the collar 50. In this regard, the transducer 52 can be inserted into the proximal end of the collar 50 at an angle, with the second pin 66 being inserted into the corresponding aperture 64. As the transducer 30 is then rotated into a final upright position, the first pin 62 is able to first enter and then translate along the slot 44. Additionally, one or both of first and second pins 62 and 66 can be made of flexible or semi-flexible material so that they can deform while the transducer 52 is inserted into the collar 50.

As illustrated in FIGS. 3 and 4A, the example catheter device 20 can include a flexible drive shaft 68. In some embodiments, the drive shaft 68 can be disposed concentrically within the catheter shaft 12 and arranged to rotate about the longitudinal axis thereof. Rotation of the drive member 68 causes the transducer 30 to pivot reciprocally about the vertical axis 53 in the fashion of a Scotch yoke mechanism, in the following manner.

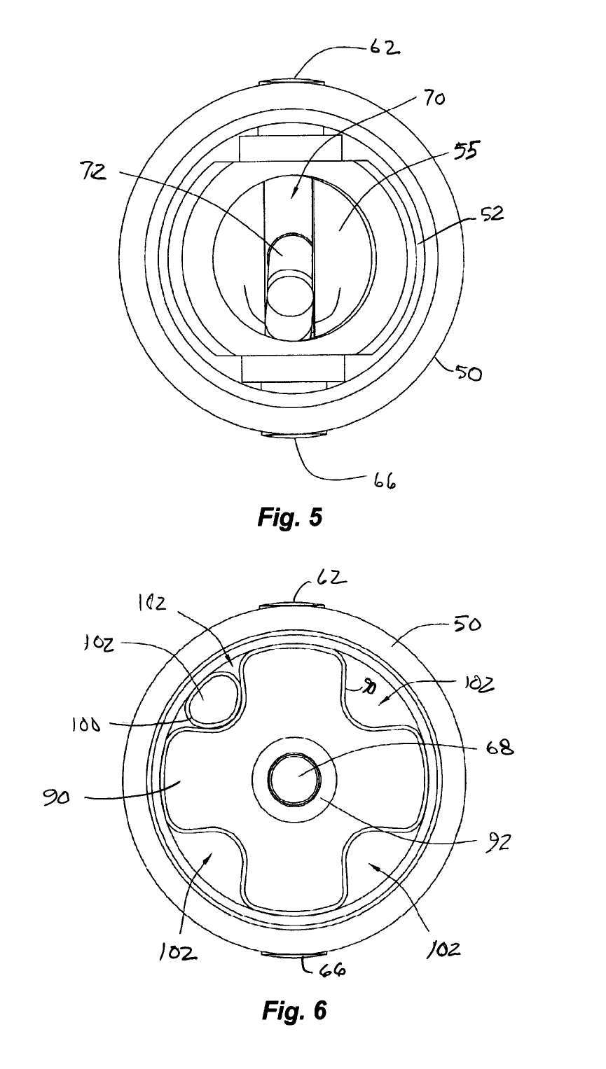

The drive shaft 68 has proximal and distal ends and generally traverses a central longitudinal axis of the catheter shaft 12. FIG. 5 is a front end elevation view of the transducer assembly 30 of FIG. 3, wherein the transducer elements 54 have been omitted to reveal a vertical slot 70 located at the back end of a housing 55 of the transducer 52. As illustrated in FIGS. 4A and 5, the distal end of the drive shaft 68 includes an arcuate portion defining a cam 72 that is engaged in the slot 70 of the transducer housing 55. The engagement between the cam 72 and the slot 70 is such that rotation of the drive shaft 68, and hence, the cam 72, causes the transducer housing 55, and hence the transducer 52, to pivot back and forth about the vertical axis 53.

In particular, the proximal end of the drive 68 can be coupled to a motor, such as a stepping motor (not illustrated) located in the handle 20. The motor can be operated to rotate the drive shaft through a selected angular increment about the long axis of the catheter shaft 12. Rotation of the drive shaft 68 causes the cam 72 to rotate conjointly. As the cam portion 72 rotates, it engages a first side wall of the slot 70, thereby causing the transducer 54 to pivot in a first direction about the vertical axis 53 defined by pins 62 and 66. At approximately 90 degrees of rotation of the cam 72 relative to the position of the cam 72 illustrated in FIG. 4, the transducer 52 will reach a maximum angular displacement in the first direction. The amount of pivotal movement of the transducer 52 can be varied by adjusting the geometries of the slot 70 and cam 72. For example, the configuration depicted in FIGS. 4A and 5 results in a pivotal movement of the transducer 32 of about 45-60 degrees in the first direction as a result of a rotation of the drive shaft 68 of about 90 degrees, As the drive shaft 68 continues to rotate to a position of between 90 and 180 degrees from its original angular position, the transducer 52 is caused to rotate back toward the central position illustrated in FIG. 5.

As the drive shaft continues to rotate between 180 and 270 degrees, the cam 72 engages a second, opposite wall of the slot 70, causing the transducer 52 to pivot in a second direction that is opposite the first direction. Maximum pivotal movement of the transducer 52 in the second direction occurs when the cam is disposed at 270 degrees relative to its original starting point. As above, the amount of pivotal movement can be varied. However, assuming symmetry in the size and arrangement of the cam 72 and slot 70, the amount of lateral pivotal movement of the transducer in the first and second directions should be substantially the same. As before, as the drive 68 rotates between 270 and 360 degrees, i.e., its original angular position, the transducer 52 returns to the central position. Thus, in the embodiment illustrated, the total pivotal movement of the transducer 52 in the transverse plane is between about 90 and 120 degrees.

FIG. 4B is a partial cross-sectional side elevation view of an alternative embodiment of a transducer assembly 30 in accordance with the present invention. The transducer assembly 30 of FIG. 4B is similar to that of the first embodiment described above in connection with FIGS. 3, 4A and 5, in that it also includes an elongated catheter shaft 12 and a pivotally reciprocating transducer 52. However, in the second embodiment, the first and second pins 62 and 55, together with their associated slot and aperture 60 and 64, have been replaced with a pair of shafts 74 (see FIG. 3, detail view A) protruding laterally from the transducer 52 and extending into corresponding openings 76 in the collar 50, such that the transducer 52 pivots vertically through a selected angle in the sagittal plane of the transducer assembly 32. Additionally, the flexible rotating drive shaft 68 and the cam 72 and slot 70 of the Scotch yoke mechanism have been replaced with a push rod 78 that moves a push member 80 into and out of engagement with a follower arm 82 extending back from the transducer housing 55.

In the example alternative embodiment of FIG. 4B, the push rod 78 can be moved reciprocally in the axial direction, as indicated by the arrow 84, by an actuator 86. In the embodiment illustrated, the actuator 86 can comprise a “shape-memory” actuator (SMA) of the types described in U.S. Pat. No. 7,658,715 to B. Park, et al., or in U.S. Prov. Pat. App. No. 61/546,419 (Attorney Docket No. 44755.827/01-0152-US), filed Oct. 12, 2011, each of which is incorporated herein by reference. In particular, the actuator 86 can comprise a pair of SMA devices 88, each of which is activated by the application of a selected voltage thereto, to alternately push the push rod 78 forward or rearward in a reciprocating manner, thereby causing the transducer 52 to pivot up and down reciprocally in the sagittal plane, as described above. Further, as will be understood by those of some skill, the pivotal motion of the transducer 30 can be made to conform to that of the first embodiment described above by the expedient of rotating the transducer assembly 30 coaxially on the catheter shaft 12 by 90 degrees. The second embodiment of FIG. 4B is advantageous in that it eliminates the elongated flexible drive shaft 68 and associated motor, and instead, disposes a relatively smaller actuator 68 entirely within the transducer assembly 30.

The catheter shaft 12, collar 50, and transducer housing 55 of either embodiment above can be made of any suitable material. An example of a material suitable for the catheter shaft 12 is engineered nylon (such as Pebax polyether block amide) and includes a tube or tubing, alternatively called a catheter tube or tubing. An example of a material suitable for the collar 50 and transducer housing 55 is 401 stainless steel and/or other material capable withstanding the frictional wear created by oscillatory movement of the transducer 52 within the transducer assembly 30.

As illustrated in FIG. 4A, in one embodiment, the catheter device 10 can include a guide 90 having a central lumen 92 extending axially therethrough. The flexible drive shaft 68 can be disposed within the central lumen 92 of the guide 90, and can be made of any suitable flexible material, such as coiled or woven stainless steel. In one embodiment, the cam 72 of the drive shaft 68 can be coated with Teflon or other lubricious material. The guide 90 can also be formed from any suitable material and can be lubricious to minimize friction with the drive shaft 68 during rotation. Examples of suitable materials include plastic, Pebax, Fluorinated Ethylene Propylene (FEP) and Teflon.

FIG. 4A further illustrates one or more optional pull tendons 94 disposed within the catheter device 10. When used, the pull tendons 94 (also called a “steering wire” or “steering line”) enable the distal end of the catheter shaft 12 to be manually articulated by a technician, typically effected under fluoroscopy. In the arrangement illustrated, a distal end of each pull tendon 94 is anchored to the distal end of the catheter shaft 12. For example, the respective distal ends of the pull tendons 94 can be affixed to the collar 50. The respective proximal ends of the pull tendons 94 are connected to the handle 20. One or more rotating actuators 96, 98 (see FIG. 1), e.g., rotatable knobs, can then be rotated to pull on respective ones of the pull tendons 94, which causes the distal end of the catheter shaft 12 to bend in a desired direction. The handle 20 and actuators 96, 98 illustrated are described in further detail in commonly owned U.S. Pat. App. No. 2008/0009745 by N. Hossack, et al., the disclosure of which is incorporated herein by reference.

The pull tendons 94 can be made of any suitable material that is of sufficient strength to withstand the pull forces initiated by actuators 96 and 98, while at the same time flexible enough to permit them to bend with the catheter shaft 12. Examples of suitable materials for the pull tendons 94 include metals (e.g., stainless steel, Nitinol or other titanium alloy) or non-metals (e.g., an aramid fiber, such as Kevlar) or other materials having good flexibility and a high tensile strength.

FIG. 6 is a cross-sectional view of the example transducer assembly 30 of FIG. 3, as seen along the lines of the section A-A taken in FIG. 4A. As illustrated in FIG. 6, the flexible drive shaft 68 is disposed concentrically within the central lumen 92 of the guide 90. The catheter device 10 can also include a tubular structure 100 which defines one or more additional lumens 102. The pull tendons 94 can be disposed in the one or more lumens 102 along substantially the entire length of the catheter shaft 12.

As discussed above, in some embodiments, a bundle of conductive wires (not illustrated) can be used to convey the respective phased actuation voltages through the handle 20 and catheter shaft 12 to respective ones of the transducer elements 54, and in a return direction, can convey the electrical signals produced by the transducer array 56 in response to its receipt of reflected ultrasound signals back through the catheter 12 to the handle 20, and thence, for example, to the control, processing and display module 14. The electrical connections of these wires to the transducer 52 need to be robust enough to withstand the oscillatory movement of the transducer 52, while also being flexible enough to permit bending proximally thereof. Examples of suitable electrical connections include coiled wires.

As another example, a flexible substrate having electrical conductors or traces disposed thereon can be used. In other embodiments, an application specific integrated circuit (ASIC) 57 is mounted on the back side of the transducer housing 55 (See, e.g., FIG. 4B). The ASIC 57 controls firing of individual transducer elements 54 in response to control signals received from the processing and control module 34. This configuration utilizes fewer conductors extending the length of the catheter shaft 12 and facilitates assembly of the connection between transducer elements 54 and the processing and control module.

As illustrated in FIG. 6, the catheter device 10 can include additional lumen 102 that run inside the catheter shaft 12 substantially parallel to the guide 90. The additional lumen 102 can be used, for example, as conduits for the conductive wire bundle (not illustrated). and/or to deliver a therapeutic device or fluid to the field isonated by the transducer 52 in order to facilitate image guided therapies or treatments. For example, a therapeutic device such as a laser fiber-bundle (not illustrated) can be used to treat tissue (e.g., an arterial occlusion) ahead of the catheter tip, either by tissue ablation, or, tissue photochemotherapy. The laser pulses can be timed with the ultrasound transmit-receive process so that the high frequency laser-induced tissue reverberations can be seen in the ultrasound image plane simultaneously. In this way, the invention can dynamically guide the operator's vision during a microsurgical procedure.

FIG. 7 is a plan view illustrating a 2D sweep pattern 700 of the transducer 52 of the example transducer assembly 30 of FIG. 4A or 4B, as may be seen, for example, in either an azimuthal or an elevational plane. As discussed above, the ultrasound “beam” emitted by the transducer 52 can be steered or “swept” through an angular field of view either “mechanically,” using either of the reciprocating pivoting mechanisms described above in connection with FIGS. 4A and 4B, or “electronically,” using the phased array technique described above in connection with FIG. 3. In the particular example embodiments of the transducer assembly 30 described above, a mechanically effected scan will result in an azimuthal sweep, whereas, an electronically effected scan of the phased array 56 will result in an elevational sweep. Of course, as discussed above, these azimuthal and elevation roles can be reversed by simply by rotating the angular position of the transducer assembly 30 on the distal end of the catheter shaft 12 by 90 degrees.

In either case, however, as the beam of the ultrasound transducer 52 is traversed through its angular motion profile, it collects data, e.g., relative angular position and distance of an object, on a line-by-line basis as the transducer 32 is repeatedly transitioned between “send” and “receive” modes. Each line 702 can be referred to as “pixel” or “line” and contains data sampled at defined positions and depths. When the transducer 52 has traversed the entire field-of-view, the set of pixels can be collected and grouped together as a “frame” representative of a 2D “slice” through the angular field of view, which is determined by the amount of angular displacement of the ultrasonic beam imparted either mechanically or electronically. In some embodiments, these two angular displacements can be approximately equal to each other and between about 90 degrees and about 120 degrees.

FIG. 8 is a perspective view illustrating a 3D sweep pattern of the transducer 52 of the two example transducer assemblies 30, showing the transducer 52 being swept both mechanically and electronically in each of two orthogonal directions, e.g., in both azimuth (A) and elevation (E). As can be seen in FIG. 8, the field of view of the transducer 52 is conical, with a circular base that subtends a segment of a sphere.

In one possible embodiment, a 3D scan can be effected by, for example, mechanically pivoting the transducer 52 to its left-most azimuthal position (−A direction), electronically effecting an elevation scan with the phased array 56 to obtain a 2D frame at that azimuthal location, then mechanically pivoting the transducer 52 in the +A direction by a small angular increment (depending on the size of the ultrasound “beam”) and obtaining a second 2D frame at the second azimuthal location adjacent to the first, and then repeating the foregoing steps until the entire field of view has been scanned. Of course, other scanning techniques can be used advantageously, e.g., right-to-left, up-to-down, with and without beam “flyback,” repeatedly imaging frames for error correction and/or resolution enhancement, and so on. In all cases, however, it desirable that the two orthogonal sweeps be synchronized with each other, e.g., with respective clocking signals applied to each of the electronic and mechanical sweep mechanisms.

The side-by-side 2D frames obtained from the foregoing procedure can be combined to form a 3D “page” of image data that, as discussed above, can be manipulated computationally to confect a wide variety of 2D images of objects located in the field of view, or if stereoscopic and/or laser holographic techniques are employed, even 3D images thereof.

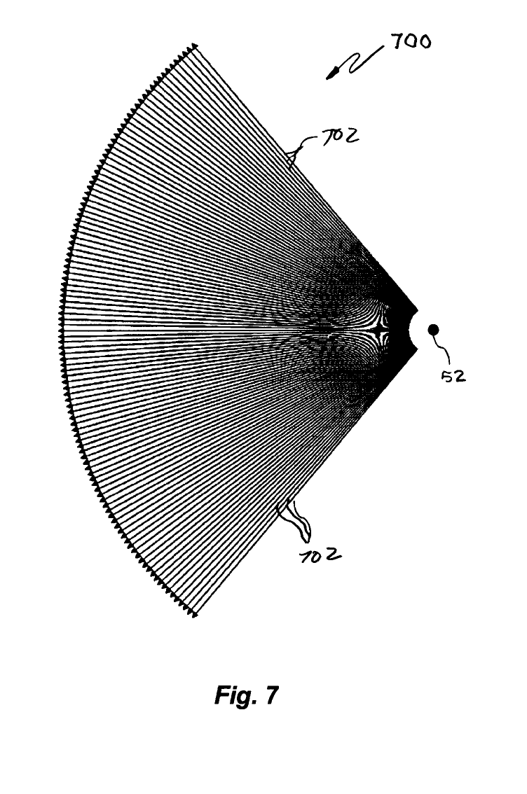

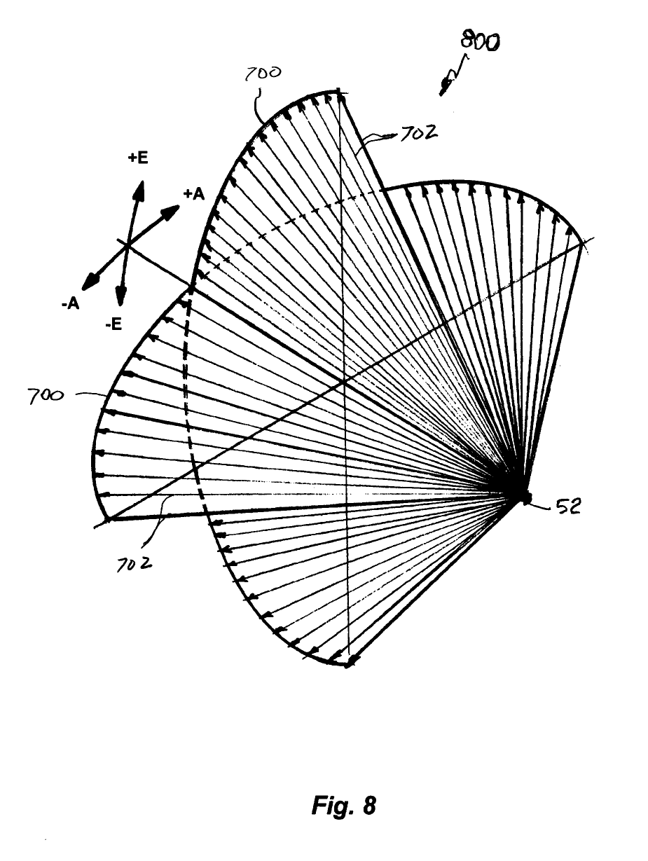

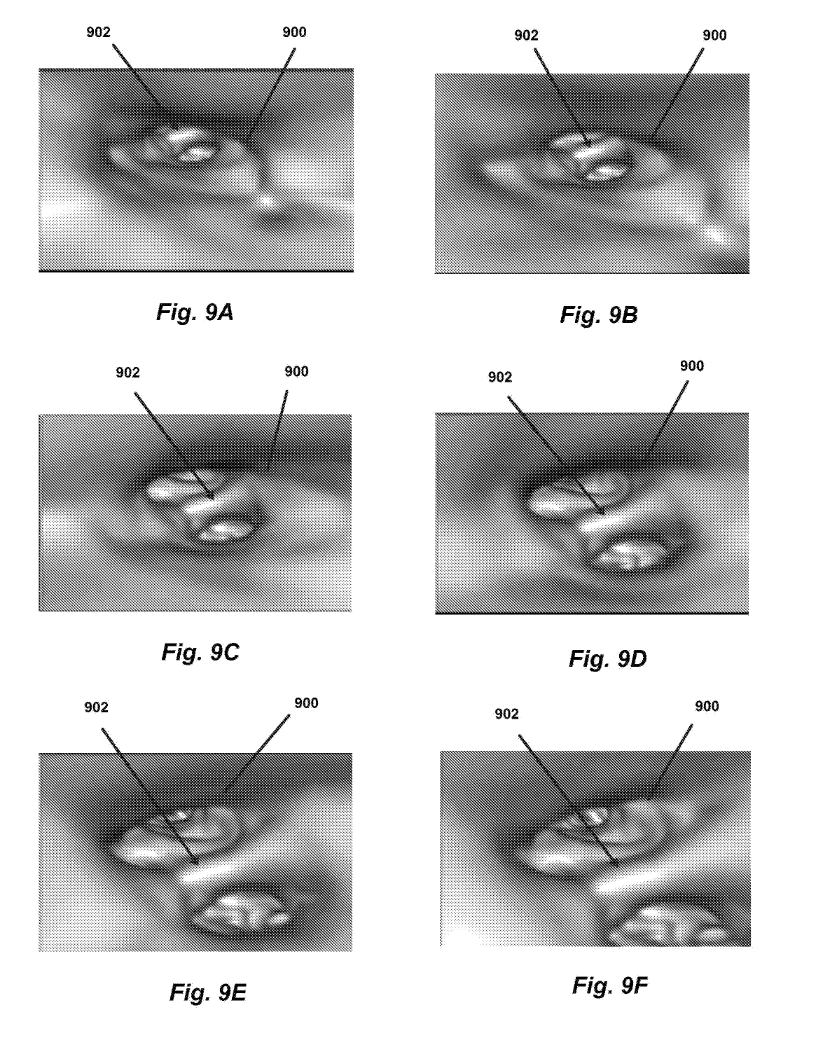

In some embodiments, the pages of 3D image data can be obtained at a rate of, for example, 15-30 pages per second. This imbues the catheter device 10 with the ability to effect a so-called “fly-through” procedure in which the distal end of the catheter device 10 is introduced into the lumen of an anatomical vessel and then advanced axially while imaging the interior of the vessel continuously and in real time, in the manner of a “movie.” FIGS. 9A-9F are images taken sequentially in the lumen 900 of a body vessel illustrating such a “fly-through” procedure. As may be seen in FIGS. 9A-9F, as the distal tip of the catheter device 10 is advanced axially through the lumen 900, the procedure enables an operator of the catheter device 10 to continuously visualize the anatomical structures in the lumen 900 ahead of the transducer assembly 502 as it is advanced toward a structure 902 that may be of particular interest, such as a lesion, a stenosis, a plaque deposit, a polyp, a heart valve, a vessel bifurcation, an aneurysm, or the like.

The foregoing procedure, which is only one of several such procedures that can be affected with the ultrasonic imaging catheter of the present invention, can thus provide the physician with a powerful tool for the diagnosis and treatment of a wide variety of cardiovascular diseases.

Although the disclosed embodiment provides a forward looking orientation for the transducer assembly, this orientation is provided for ease of explanation and is not limiting to the application of the current concepts. For example, a movable transducer assembly containing multiple transducers can be oriented perpendicular to the longitudinal axis in a side looking orientation and still employ the teachings of the present concept. Similarly, the movable transducer assembly can be oriented at any angle with respect to the longitudinal axis to provide the desired image section beyond the catheter.

Systems and their associated components have been described herein above with reference to example embodiments of the invention, including their structures and techniques. In view of the many possible embodiments to which the principles of this invention can be applied, it should be recognized that the embodiments described herein with respect to the drawing figures are meant to be illustrative only and should not be taken as limiting the scope of invention. Therefore, the invention as described herein contemplates all such embodiments as may come within the scope of the following claims and the functional equivalents thereof.

What is claimed is:

1. A catheter device, comprising:
an elongated catheter shaft;
a forward looking ultrasonic transducer disposed at a distal end of the catheter shaft;
a first device for sweeping an ultrasonic beam produced by the transducer in a first plane and through a first angle therein; and
a second device for sweeping the ultrasonic beam in a second plane and through a second angle therein, the second plane being disposed orthogonally to the first plane.

2. The catheter device of claim 1, wherein at least one of the first and the second devices comprises a mechanism for converting rotational movement into reciprocating linear motion.

3. The catheter device of claim 2, wherein the mechanism comprises a Scotch yoke.

4. The catheter device of claim 1, wherein at least one of the first and the second devices comprises:
an elongated flexible drive shaft disposed in a lumen of the catheter shaft;
a cam disposed at a distal end of drive shaft and engaged in an elongated slot disposed in a proximal end of the transducer; and
a motor rotatably coupled to a proximal end of the flexible shaft.

5. The catheter device of claim 4, wherein the motor comprises a stepping motor.

6. The catheter device of claim 4, wherein the catheter device further comprises a handle, and the motor is disposed within the handle.

7. The catheter device of claim 1, wherein at least one of the first and the second devices comprises a shape-memory actuator (SMA) having a push rod with a push member disposed at a distal end thereof and in engagement with a follower arm extending proximally from the transducer.

8. The catheter device of claim 1, wherein:
the transducer comprises a linear phased array transducer; and
at least one of the first and the second devices comprises circuitry for electronically sweeping the ultrasonic beam.

9. The catheter device of claim 8, wherein:
the transducer comprises a plurality of transducer elements; and
at least one of the transducer elements comprises lead zirconate titanate (PZT).

10. The catheter device of claim 1, wherein the first and second angles are approximately equal to each other.

11. The catheter device of claim 1, wherein at least one of the first and the second angles is between about 90 degrees and about 120 degrees.

12. A method for acquiring a page of three-dimensional (3D) image data corresponding to a selected field of view using the catheter device of claim 1, the method comprising:
sweeping the ultrasonic beam through the first angle in the first plane using the first device to form a first frame of two-dimensional (2D) image data;
incrementing the angular position of the beam by a selected amount in the second plane using the second device;
sweeping the beam through the first angle in the first plane using the first device to form a second frame of two-dimensional (2D) image data spatially adjacent to the first frame;
repeating the preceding steps until the entire field of view has been scanned by the beam; and
combining the 2D image data frames to form a page of 3D image data corresponding to the selected field of view.

13. The method of claim 12, wherein the selected amount is about the same as a width of the ultrasonic beam.

14. The method of claim 12, wherein the first plane comprises an azimuthal plane and the second plane comprises an elevational plane.

15. The method of claim 12, wherein the first plane comprises an elevational plane and the second plane comprises an azimuthal plane.

16. A method for acquiring a page of three-dimensional (3D) image data corresponding to a selected field of view, the method comprising:
providing an elongated catheter shaft having a forward looking linear phased array ultrasonic transducer disposed at a distal end thereof;
coupling a first device to the transducer for mechanically sweeping an ultrasonic beam produced by the transducer in a first plane and through a first angle therein;
coupling a second device to the transducer for electronically sweeping the ultrasonic beam in a second plane and through a second angle therein, the second plane being orthogonal to the first plane;
scanning the entire field of view with the ultrasonic beam using the first and second devices to form a plurality of frames of two-dimensional (2D) image data; and
combining the 2D image data frames to form a page of three-dimensional (3D) image data corresponding to the selected field of view.

17. The method of claim 16, further comprising:
processing the 3D image data to render at least one data image therefrom; and
displaying the rendered data image on a display device.

18. The method of claim 16, wherein the scanning comprises:
intermittently pulsing the ultrasound beam;
receiving reflections of the beam from structures disposed in the field of view between the pulses; and
converting the reflections to image data.

19. The method of claim 16, wherein the field of view comprises an interior of a vessel or a chamber in a patient's body.

20. A method for performing a fly-through procedure in a vasculature of a patient's body, the method comprising:
providing an elongated catheter shaft having:
a forward looking linear phased array ultrasonic transducer disposed at a distal end thereof;
a first device coupled to the transducer for mechanically sweeping an ultrasonic beam produced by the transducer in a first plane and through a first angle therein;
a second device coupled to the transducer for electronically sweeping the ultrasonic beam in a second plane and through a second angle therein, the second plane being orthogonal to the first plane;
inserting the distal end of the catheter shaft into a lumen of the vasculature;
scanning the interior of the vasculature with the ultrasonic beam using the first and second devices to form a plurality of frames of two-dimensional (2D) image data;
combining the 2D image data frames to form a page of three-dimensional (3D) image data corresponding to the interior of the vasculature;
processing the 3D image data to render a 2D image of the interior of the vessel;
displaying the 2D image on a display;
advancing the distal end of the catheter shaft further distally into the lumen; and
repeating the scanning, combining, processing, displaying and advancing such that a continuous image of the interior of the vasculature in a region ahead of the distal end of the catheter is displayed on the display as the distal end of the catheter is advanced through the lumen.
